(19) United States
(12) Patent Application Publication
(10) Pub. No.: US 2023/0110652 A1
(43) Pub. Date: Apr. 13, 2023

(54) BRAIN DATA VISUALIZATION

(71) Applicant: Omniscient Neurotechnology Pty Limited, Sydney (AU)

(72) Inventors: Stephane Philippe Doyen, Mount Kuring Gai (AU); Michael Edward Sughrue, Brighton Le Sands (AU); Hugh Monro Taylor, Point Piper (AU)

(21) Appl. No.: 17/963,151

(22) Filed: Oct. 10, 2022

Related U.S. Application Data
(60) Provisional application No. 63/255,411, filed on Oct. 13, 2021.

(51) Int. Cl. A61B 5/00; A61B 5/055

(52) U.S. Cl. CPC A61B 5/002; A61B 5/055

(57) ABSTRACT

Methods, systems, and apparatus, including computer programs encoded on computer storage media, for determining and visualizing contribution values of different brain regions to a medical condition. One of the methods includes receiving brain data for a brain of a patient, processing the brain data to determine a partition of the data into a plurality of brain parcellation pairs, receiving an indication of a medical condition, determining a contribution value for at least some of the plurality of brain parcellation pairs, where the contribution value characterizes a contribution of the brain parcellation pair to the medical condition, and providing the contribution values for display on a user computing device.

### CROSS-REFERENCE TO RELATED APPLICATIONS

This application claims the benefit under 35 U.S.C. § 119(e) of the filing date of U.S. Patent Application No. 63/255,411, for PERSONAL BRAIN DATA VISUALIZATION, which was filed on Oct. 13, 2021, and which is incorporated here by reference.

### TECHNICAL FIELD

The present disclosure generally relates to using machine learning on medical imaging data.

### BACKGROUND

Medical imaging includes the technique and process of creating visual representations of the interior of a body for clinical analysis and medical intervention, as well as visual representation of the function of some organs or tissues (physiology). Medical imaging seeks to reveal internal structures hidden by the skin and bones, as well as to diagnose and treat disease. Medical imaging also establishes a database of normal anatomy and physiology.

### SUMMARY

This specification describes a method for generating visualization of contributions of different brain parcellation or pairs of parcellations of a patient to a symptom, a condition, e.g., a medical condition, a behavior, or a trait. The method includes receiving brain data for a brain of a patient, processing the brain data to determine a partition of the data into a plurality of brain parcellation pairs, receiving an indication of a medical condition, determining a contribution value for each brain parcellation pair, where the contribution value characterizes a contribution of the brain parcellation pair to the medical condition, and providing the contribution values for display on a user computing device.

The brain data can characterize a connectivity in the brain of the patient and/or brain activity patterns in the brain of the patient, and can be obtained by processing an image of the brain. The image of the brain can be obtained using any suitable medical imaging technique, e.g., Magnetic Resonance Imaging (MRI), functional Magnetic Resonance Imaging (fMRI), functional Near-Infrared Spectroscopy (fNIRS), Magnetoencephalography (MEG), Electroencephalography (EEG), Diffusion Tensor Imaging (DTI), or any other appropriate imaging modality. Furthermore, a machine learning model can be used to process the brain data and determine a prediction that characterizes a likelihood of whether the patient has the symptom, the medical condition, the behavior, or the trait.

The method described in this specification utilizes the brain data and the prediction generated by the machine learning model to facilitate a visualization of contributions of individual brain parcellation pairs to the predicted outcome. A parcellation is a volume/region of a brain of a patient and typically has specified functional, cytological and/or structural characteristics. The number of parcellations making up a brain can be more than 50, more than 100, more than 250, more than 350, more than 500, or more than 1000. The volume of the parcellations can range (or be the same) where no individual parcellation is smaller than a cubic millimeter, smaller than 50 cubic millimeters, smaller than 100 cubic millimeters, or smaller than 500 cubic millimeters and/or where no individual parcellation is larger than ¼ of a brain hemisphere, larger than a ⅙ of a brain hemisphere, larger than a 1/12 of a brain hemishphere, or larger than 2 cubic centimeters. For example, parcellations can range in size from 50 cubic millimeters to 2 cubic centimeters and range in number between 100 and 400. Such parcellations do not need to be (but can be) uniform in volume and/or shape. Determining and visualizing different contributions to the outcome by individual brain parcellation pairs helps to explain the prediction generated by the machine learning model and enables clinicians to design an effective treatment plan.

According to a first aspect, there is provided a method that includes: receiving brain data for a brain of a patient, processing the brain data to determine a partition of the data into a plurality of brain parcellation pairs, receiving an indication of a medical condition, determining a contribution value for at least some of the plurality of brain parcellation pairs, wherein the contribution value characterizes a contribution of the brain parcellation pair to the medical condition, and providing the contribution values for display on a user computing device.

In some implementations, the contribution value is a SHAP value, and wherein determining the contribution value for each brain parcellation pair comprises determining the SHAP value for each brain parcellation pair using SHAP methodology.

In some implementations, the method further includes providing, on a user computing device, a visualization that compares the respective contributions of the plurality of brain parcellation pairs to the medical condition based on the respective contribution values.

In some implementations, the method further includes processing the brain data of the brain of the patient to determine a connectivity value for each of the brain parcellation pairs of the patient, wherein the connectivity value for the brain parcellation pair characterizes blood flow or blood oxygen level over time in the regions of the brain represented by the brain parcellation pair (the blood flow acting as a proxy for electrical activity), and determining, for each brain parcellation pair of the patient, a position of the connectivity value of the brain parcellation pair of the patient within either a first distribution of connectivity values for patients with a medical condition or a second distribution of connectivity values for patients without the medical condition.

In some implementations, the first distribution of connectivity values is specified for the brain parcellation pair across a population having the medical condition, and the second distribution of connectivity values is specified for the brain parcellation pair across a population not having the medical condition.

In some implementations, the method further includes providing, on a user computing device, a visualization that indicates the position of the patient's connectivity value for a parcellation pair of interest within the first distribution or the second distribution.

In some implementations, processing the brain data of the brain to determine the partition of brain data into the plurality of brain parcellation pairs comprises generating a connectivity matrix that characterizes a connectivity in the brain of the patient.

In some implementations, the combination of the respective contribution values of all brain parcellation pairs represents a probability score that characterizes an overall likelihood of the patient having the medical condition.

In some implementations, the probability score is determined by using a trained machine learning model that is configured to process an input derived from the brain data of the brain of the patient and generate the probability score.

According to a second aspect, there is provided a method that includes: receiving brain data for a brain of a patient, the brain data comprising a plurality of brain parcellation pairs, receiving an indication of a symptom, obtaining a model for processing the brain data to predict whether the patient has the symptom, determining a contribution value for each brain parcellation pair in the plurality of brain parcellation pairs, wherein the contribution value characterizes a contribution of the brain parcellation pair to the symptom, the contribution value based at least in part on the brain data and the model, and providing contribution values that meet a criteria for display by a user computer.

In some implementations, the contribution value is a SHAP value, and determining the contribution value for each brain parcellation pair includes determining the SHAP value for each brain parcellation pair using SHAP methodology.

In some implementations, the criteria specifies a number of top-contributing brain parcellation pairs based on the magnitudes of the contribution values, and wherein the method further comprises providing the number of brain parcellation pairs and the respective contribution values for display by a user computer.

In some implementations, the criteria specifies a threshold magnitude contribution value, and wherein the method further comprises providing the contribution values above the threshold, and the respective brain parcellation pairs, for display by a user computer.

According to a third aspect, there is provided a system including: one or more computers; and one or more storage devices communicatively coupled to the one or more computers, where the one or more storage devices store instructions that, when executed by the one or more computers, cause the one or more computers to perform the operations of the method of any preceding aspect.

According to a fourth aspect, there are provided one or more non-transitory computer storage media storing instructions that when executed by one or more computers cause the one or more computers to perform the operations of the method of any preceding aspect. The subject matter described in this specification can be implemented in particular embodiments so as to realize one or more of the following advantages.

A symptom or behavior expression can be identified (and in some implementations quantified) from brain data using machine learning. In particular, systems and methods described in this specification can identify, from small samples and high dimensional data, parcellations pairs of the brain that contribute to symptoms or behaviors, providing insights on complex relations and magnitudes and dimensionality of predictors. The embodiments described allow objective measurement of the parcellation pairs primarily responsible for a particular symptom or behavior.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1A and FIG. 1B are block diagrams that illustrate an example computer system for use in processing medical images.

FIG. 2 is a block diagram of an example system for determining contributions of brain regions to a medical condition.

FIG. 3 is a flow diagram of an example process for determining contributions of brain regions to a medical condition.

FIG. 4 illustrates an example graphical user interface displaying a visualization of contributions of different parcellation pairs to a medical condition.

Like reference numbers and designations in the various drawings indicate like elements.

### DETAILED DESCRIPTION

This specification describes a method for determining and visualizing contributions of different brain pairs of parcellations of the brain of a patient to a predicted condition, e.g., depression, anxiety, schizophrenia, or any other appropriate condition, e.g., medical condition, behavior, trait, or symptom. A machine learning model can be used to process brain data and determine a prediction score that characterizes a likelihood that the patient has the condition. Based on the prediction score and the brain data, the method can determine contribution values for different pairs of parcellations that characterize their respective contributions to the medical condition.

The brain data can be obtained by, e.g., processing an image of the brain of the patient (e.g., a Magnetic Resonance Image (MM), or any other appropriate type of image) and determining a partition of the brain into a plurality of brain parcellation pairs. A brain “parcel” is used interchangeably with brain “parcellation” and a “brain parcellation pair” refers to a pair of such brain parcels. The brain parcels can represent structurally, cytologically and/or functionally distinct regions of the brain. Parcel connectivity can be represented by, e.g., a connectivity matrix that characterizes a connectivity (e.g., synaptic connectivity between neurons, amount and health of nerve tracts between specified parcels, correlation of activity of pairs of parcels, or any other appropriate biological element).

The method described in this specification can process data defining the brain parcellation pairs (e.g., the connectivity matrices) and determine contribution values for each of the brain parcellation pairs that characterize a contribution of each of the pairs to the predicted condition (e.g., the prediction determined by the machine learning model). In one example, the contribution value can be a SHAP value that is a numerical value determined by using the SHAP (SHapley Additive exPlanations) methodology. However, the method described in this specification is not limited to the SHAP methodology, and any other appropriate methodology can also be used to determine the contribution of each brain parcellation pair to the medical condition, e.g., any methodology that can determine feature importance at an individual level.

The method can provide the respective contribution values of the brain parcellation pairs (e.g., the top ten contributing pairs, or any other appropriate number of pairs) as explainability data that explains the medical condition, e.g., that explains which brain regions, represented by the brain parcellation pairs, contribute the most (or the least) to the medical condition. Example systems that can perform the aforementioned method will be described in more detail next.

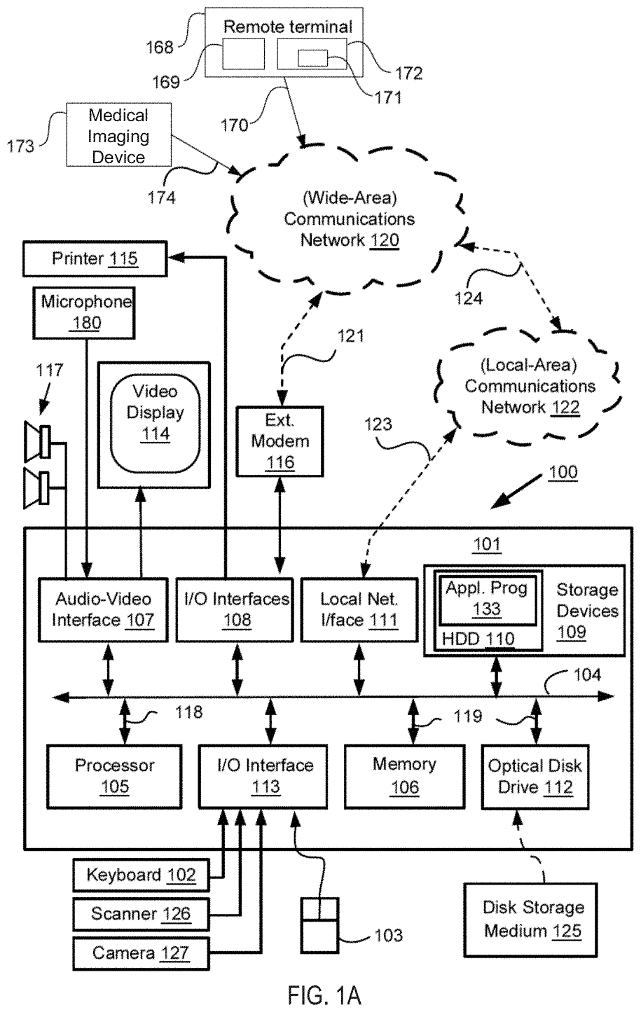

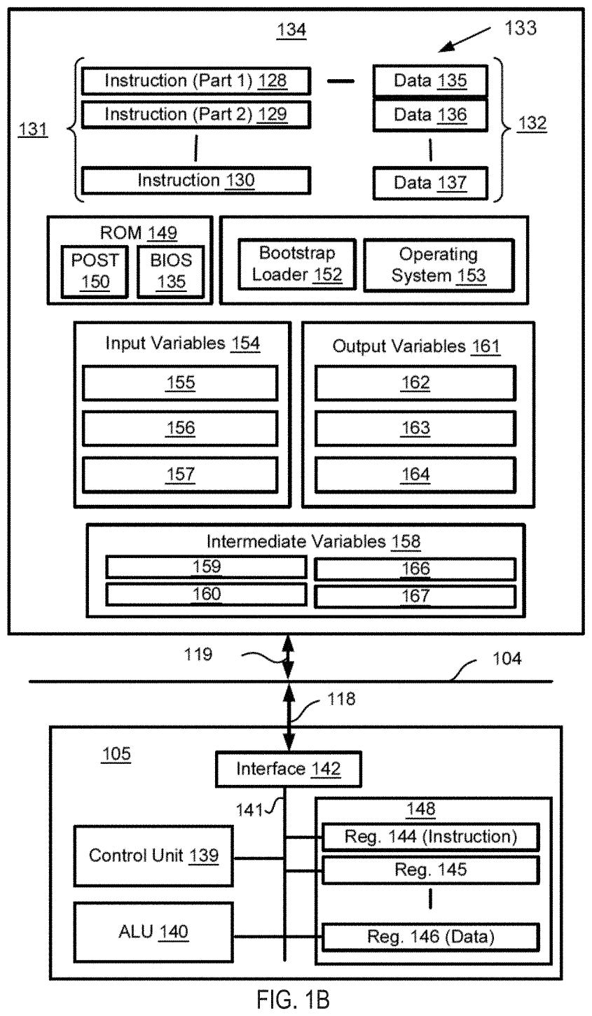

FIGS. 1A and 1B are block diagrams of a computer system 100 upon which one can practice arrangements described in this specification. The following description is directed primarily to a computer server module 101. However, the description applies equally or equivalently to one or more remote terminals 168.

As seen in FIG. 1A, the computer system 100 includes: the server computer module 101; input devices such as a keyboard 102, a pointer device 103 (e.g., a mouse), a scanner 126, a camera 127, and a microphone 180; and output devices including a printer 115, a display device 114 and loudspeakers 117. An external Modulator-Demodulator (Modem) transceiver device 116 may be used by the computer server module 101 for communicating to and from the remote terminal 168 over a computer communications network 120 via a connection 121 and a connection 170. The aforementioned communication can take place between the remote terminal 168 and “the cloud” which in the present description comprises at least the one server module 101. The remote terminal 168 typically has input and output devices (not shown) which are similar to those described in regard to the server module 101. The communications network 120 may be a wide-area network (WAN), such as the Internet, a cellular telecommunications network, or a private WAN. Where the connection 121 is a telephone line, the modem 116 may be a traditional “dial-up” modem. Alternatively, where the connection 121 is a high capacity (e.g., cable) connection, the modem 116 may be a broadband modem. A wireless modem may also be used for wireless connection to the communications network 120. The computer server module 101 typically includes at least one processor unit 105, and a memory unit 106. For example, the memory unit 106 may have semiconductor random access memory (RAM) and semiconductor read only memory (ROM). The remote terminal 168 typically includes at least one processor 169 and a memory 172. The computer server module 101 also includes a number of input/output (I/O) interfaces including: an audio-video interface 107 that couples to the video display 114, loudspeakers 117 and microphone 180; an I/O interface 113 that couples to the keyboard 102, mouse 103, scanner 126, camera 127 and optionally a joystick or other human interface device (not illustrated); and an interface 108 for the external modem 116 and printer 115. In some implementations, the modem 116 may be incorporated within the computer module 101, for example within the interface 108. The computer module 101 also has a local network interface 111, which permits coupling of the computer system 100 via a connection 123 to a local-area communications network 122, known as a Local Area Network (LAN). As illustrated in FIG. 1A, the local communications network 122 may also couple to the wide network 120 via a connection 124, which would typically include a so-called “firewall” device or device of similar functionality. The local network interface 111 may include an Ethernet circuit card, a Bluetooth® wireless arrangement or an IEEE 802.11 wireless arrangement; however, numerous other types of interfaces may be practiced for the interface 111.

The I/O interfaces 108 and 113 may afford either or both of serial or parallel connectivity; the former may be implemented according to the Universal Serial Bus (USB) standards and having corresponding USB connectors (not illustrated). Storage memory devices 109 are provided and typically include a hard disk drive (HDD) 110. Other storage devices such as a floppy disk drive and a magnetic tape drive (not illustrated) may also be used. An optical disk drive 112 is typically provided to act as a non-volatile source of data. Portable memory devices, optical disks (e.g., CD-ROM, DVD, Blu-ray Disc™), USB-RAM, portable, external hard drives, and floppy disks, for example, may be used as appropriate sources of data to the system 100.

The components 105 to 113 of the computer module 101 typically communicate via an interconnected bus 104 and in a manner that results in a conventional mode of operation of the computer system 100 known to those in the relevant art. For example, the processor 105 is coupled to the system bus 104 using a connection 118. Likewise, the memory 106 and optical disk drive 112 are coupled to the system bus 104 by connections 119.

The techniques described in this specification may be implemented using the computer system 100, e.g., may be implemented as one or more software application programs 133 executable within the computer system 100. In some implementations, the one or more software application programs 133 execute on the computer server module 101 (the remote terminal 168 may also perform processing jointly with the computer server module 101), and a browser 171 executes on the processor 169 in the remote terminal, thereby enabling a user of the remote terminal 168 to access the software application programs 133 executing on the server 101 (which is often referred to as “the cloud”) using the browser 171. In particular, the techniques described in this specification may be implemented by instructions 131 (see FIG. 1B) in the software 133 that are carried out within the computer system 100. The software instructions 131 may be formed as one or more code modules, each for performing one or more particular tasks. The software may also be divided into two separate parts, in which a first part and the corresponding code modules perform the described techniques and a second part and the corresponding code modules manage a user interface between the first part and the user. The software may be stored in a computer readable medium, including the storage devices described below, for example. The software is loaded into the computer system 100 from the computer readable medium, and then executed by the computer system 100. A computer readable medium having such software or computer program recorded on the computer readable medium is a computer program product. Software modules that execute techniques described in this specification may also be distributed using a Web browser.

The software 133 is typically stored in the HDD 110 or the memory 106 (and possibly at least to some extent in the memory 172 of the remote terminal 168). The software is loaded into the computer system 100 from a computer readable medium, and executed by the computer system 100. Thus, for example, the software 133, which can include one or more programs, may be stored on an optically readable disk storage medium (e.g., CD-ROM) 125 that is read by the optical disk drive 112. A computer readable medium having such software or computer program recorded on it is a computer program product.

In some instances, the application programs 133 may be supplied to the user encoded on one or more CD-ROMs 125 and read via the corresponding drive 112, or alternatively may be read by the user from the networks 120 or 122. Still further, the software can also be loaded into the computer system 100 from other computer readable media. Computer readable storage media refers to any non-transitory tangible storage medium that provides recorded instructions and/or data to the computer system 100 for execution and/or processing. Examples of such storage media include floppy disks, magnetic tape, CD-ROM, DVD, Blu-ray™ Disc, a hard disk drive, a ROM or integrated circuit, USB memory, a magneto-optical disk, or a computer readable card such as a PCMCIA card and the like, whether or not such devices are internal or external of the computer module 101. Examples of transitory or non-tangible computer readable transmission media that may also participate in the provision of software, application programs, instructions and/or data to the computer module 101 include radio or infra-red transmission channels as well as a network connection to another computer or networked device, and the Internet or Intranets including e-mail transmissions and information recorded on Websites and the like.

The second part of the application programs 133 and the corresponding code modules mentioned above may be executed to implement one or more graphical user interfaces (GUIs) to be rendered or otherwise represented upon the display 114. For example, through manipulation of the keyboard 102 and the mouse 103, a user of the computer system 100 and the application may manipulate the interface in a functionally adaptable manner to provide controlling commands and/or input to the applications associated with the GUI(s). Other forms of functionally adaptable user interfaces may also be implemented, such as an audio interface utilizing speech prompts output via the loudspeakers 117 and user voice commands input via the microphone 180.

FIG. 1B is a detailed schematic block diagram of the processor 105 and a “memory” 134. The memory 134 represents a logical aggregation of all the memory modules (including the HDD 109 and semiconductor memory 106) that can be accessed by the computer module 101 in FIG. 1A.

When the computer module 101 is initially powered up, a power-on self-test (POST) program 150 can execute. The POST program 150 can be stored in a ROM 149 of the semiconductor memory 106 of FIG. 1A. A hardware device such as the ROM 149 storing software is sometimes referred to as firmware. The POST program 150 examines hardware within the computer module 101 to ensure proper functioning and typically checks the processor 105, the memory 134 (109, 106), and a basic input-output systems software (BIOS) module 151, also typically stored in the ROM 149, for correct operation. Once the POST program 150 has run successfully, the BIOS 151 can activate the hard disk drive 110 of FIG. 1A. Activation of the hard disk drive 110 causes a bootstrap loader program 152 that is resident on the hard disk drive 110 to execute via the processor 105. This loads an operating system 153 into the RAM memory 106, upon which the operating system 153 commences operation. The operating system 153 is a system level application, executable by the processor 105, to fulfil various high-level functions, including processor management, memory management, device management, storage management, software application interface, and generic user interface.

The operating system 153 manages the memory 134 (109, 106) to ensure that each process or application running on the computer module 101 has sufficient memory in which to execute without colliding with memory allocated to another process. Furthermore, the different types of memory available in the system 100 of FIG. 1A must be used properly so that each process can run effectively. Accordingly, the aggregated memory 134 is not intended to illustrate how particular segments of memory are allocated (unless otherwise stated), but rather to provide a general view of the memory accessible by the computer system 100 and how such is used.

As shown in FIG. 1B, the processor 105 includes a number of functional modules including a control unit 139, an arithmetic logic unit (ALU) 140, and a local or internal memory 148, sometimes called a cache memory. The cache memory 148 typically includes a number of storage registers 144-146 in a register section. One or more internal busses 141 functionally interconnect these functional modules. The processor 105 typically also has one or more interfaces 142 for communicating with external devices via the system bus 104, using a connection 118. The memory 134 is coupled to the bus 104 using a connection 119.

The application program 133 includes a sequence of instructions 131 that may include conditional branch and loop instructions. The program 133 may also include data 132 which is used in execution of the program 133. The instructions 131 and the data 132 are stored in memory locations 128, 129, 130 and 135, 136, 137, respectively. Depending upon the relative size of the instructions 131 and the memory locations 128-130, a particular instruction may be stored in a single memory location as depicted by the instruction shown in the memory location 130. Alternatively, an instruction may be segmented into a number of parts each of which is stored in a separate memory location, as depicted by the instruction segments shown in the memory locations 128 and 129.

In general, the processor 105 is given a set of instructions which are executed therein. The processor 105 waits for a subsequent input, to which the processor 105 reacts to by executing another set of instructions. Each input may be provided from one or more of a number of sources, including data generated by one or more of the input devices 102, 103, data received from an external source 173, e.g., a medical imaging device 173 such as an MM or DTI scanner, X-ray, ultrasound or other medical imaging device across one of the networks 120, 122, data retrieved from one of the storage devices 106, 109 or data retrieved from a storage medium 125 inserted into the corresponding reader 112, all depicted in FIG. 1A. The execution of a set of the instructions may in some cases result in output of data. Execution may also involve storing data or variables to the memory 134.

Some techniques described in this specification use input variables 154, e.g., data sets characterizing one or more anatomical or surgical structures, which are stored in the memory 134 in corresponding memory locations 155, 156, 157. The techniques can produce output variables 161, which are stored in the memory 134 in corresponding memory locations 162, 163, 164. Intermediate variables 158 may be stored in memory locations 159, 160, 166 and 167.

Referring to the processor 105 of FIG. 1B, the registers 144, 145, 146, the arithmetic logic unit (ALU) 140, and the control unit 139 work together to perform sequences of micro-operations needed to perform “fetch, decode, and execute” cycles for every instruction in the instruction set making up the program 133. Each fetch, decode, and execute cycle can include i) a fetch operation, which fetches or reads an instruction 131 from a memory location 128, 129, 130; ii) a decode operation in which the control unit 139 determines which instruction has been fetched; and iii) an execute operation in which the control unit 139 and/or the ALU 140 execute the instruction.

Thereafter, a further fetch, decode, and execute cycle for the next instruction may be executed. Similarly, a store cycle may be performed by which the control unit 139 stores or writes a value to a memory location 132.

Each step or sub-process in the techniques described in this specification may be associated with one or more segments of the program 133 and is performed by the register section 144, 145, 146, the ALU 140, and the control unit 139 in the processor 105 working together to perform the fetch, decode, and execute cycles for every instruction in the instruction set for the noted segments of the program 133. Although a cloud-based platform has been described for practicing the techniques described in this specification, other platform configurations can also be used. Furthermore, other hardware/software configurations and distributions can also be used for practicing the techniques described in this specification.

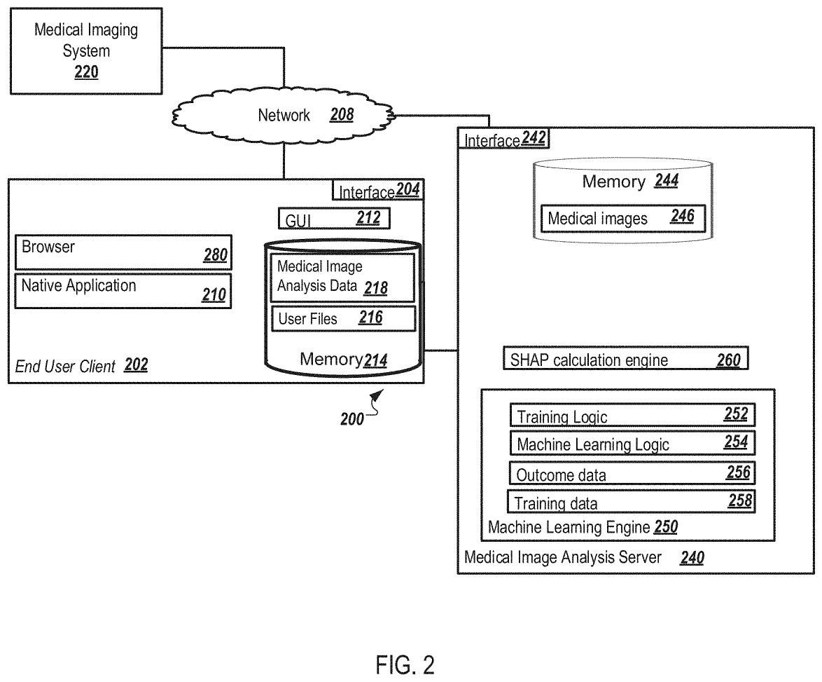

FIG. 2 is a block diagram illustrating an example system 200 for determining and visualizing contributions of different brain regions, e.g., parcellations, of the brain of a patient to a condition, a symptom, a behavior, or a trait. The system of FIG. 2 may be implemented within a computer system as described with reference to FIGS. 1A and 1B. Specifically, the illustrated system 200 includes or is communicably coupled with a Medical Image Analysis server 240, an end-user client device 202, a network 208 (which can include a local area network (LAN), a wide area network (WAN), the Internet, or a combination thereof), and a medical imaging system 220. Although shown separately, in some implementations, functionality of two or more systems, devices, or servers may be provided by a single system or server. In some implementations, the functionality of one illustrated system, server, or engine may be provided by multiple systems, servers, or engines, respectively.

An end-user client device 202 (also referred to herein as client device 202 or device 202) is an electronic device that is capable of requesting and receiving content over the network 208. The end-user client device 202 can include any client computing device such as a laptop/notebook computer, wireless data port, smart phone, personal data assistant (PDA), tablet computing device, one or more processors within these devices, or any other suitable processing device that can send and receive data over the network 208. For example, the end-user client device 202 can include, e.g., a computer that includes an input device, such as a keypad, touch screen, or other device that can accept user information, and an output device that conveys information, e.g., associated with the operation of the Medical Image Analysis server 240, or the client device itself, including digital data, visual information, or the GUI 212. The end-user client device 202 can include one or more client applications (as described above). A client application is any type of application that allows the end-user client device 202 to request and view content on a respective client device. In some implementations, a client application can use parameters, metadata, and other information received, e.g., at launch, to access a particular set of data from the Medical Image Analysis server 240. In some instances, a client application may be an agent or client-side version of the one or more enterprise applications running on an enterprise server (not shown).

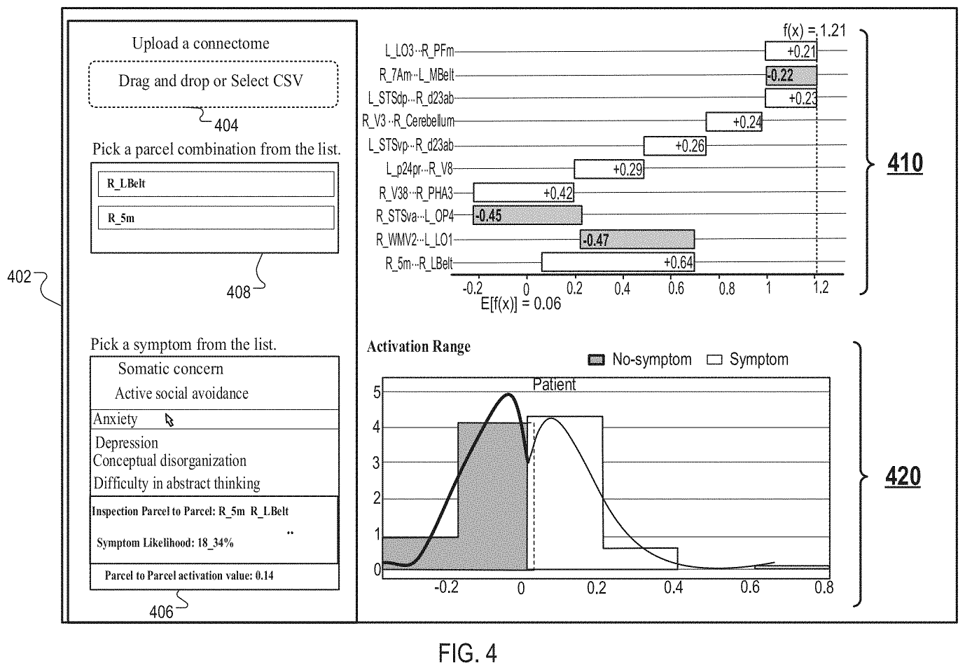

The end-user client device 202 typically includes one or more applications, such as a browser 280 or a native application 210, to facilitate sending and receiving of content over the network 108. Examples of content presented at a client device 202 include images from medical imaging system 220, and visualization of contributions of different brain regions to a medical condition, e.g., as shown in FIG. 4.

Medical imaging system 220 can be any appropriate imaging system, for example an MRI system, CT system, X-ray system, EEG system or NIRS system. In an implementation, the medical imaging system may be a functional MM (fMRI) imaging system to produce resting state fMRI images of the brain. In other examples the imaging data may selected from at least one of, Magnetoencephalograph (MEG), electroencephalograph (EEG), magnetic resonance imaging (MRI), and diffusion tensor imaging (DTI). While only one medical imaging system 220 is shown in FIG. 2 images can be received from one or more medical imaging systems.

An end user of the end-user client device 202 can provide an input to the Medical Image Analysis server 240 through a graphical user interface (GUI) 212. For example, the user can use a machine learning engine 250 included in the server 240 to carry out one or more tasks associated with analyzing one or more medical images. These tasks can include, e.g., processing one or more images of the brain obtained by the medical imaging system 220 to generate brain data that characterizes structural/functional connectivity of the brain of the patient. The tasks can further include, e.g., processing the brain data to generate a prediction (e.g., an outcome data 256) that characterizes a likelihood that the patient has a particular condition, e.g., symptom, behavior, or trait.

The user input can include, e.g., one or more selections of a series of medical images 246, e.g. fMRI images to make a measurement of functional and/or structural data, for example a fMRI image processed to show a connectomic map of the brain of a subject suffering from, or displaying, a particular set of symptoms or behaviors. In another example, the series of images may be selected automatically by the machine learning engine 250. Once the end user provides the input, the machine learning engine 250 of the Medical Image Analysis server 240 can process the data associated with the user input to determine a likelihood 252 that particular data derived from a brain activity sensing system, e.g., a connectivity matrix derived from the medical images 246, is associated with a particular behavior or symptom. A connectivity matrix characterizes the strength of connections between different brain regions, e.g., between parcels. In one embodiment, there can be hundreds of parcels, e.g., 379 parcels, resulting in tens of thousands of unique matrix elements, e.g., more than 1,000, more than 5,000, more than 10,000, more than 30,000 or more than 70,000 unique matrix elements.

The machine learning engine 250 can include a training logic 252 used to train machine learning logic 254 to identify one or more behaviors or symptoms associated with particular structures or variables in the series of medical images.

Machine learning engine 250 can further include machine learning logic 254. Machine learning logic 254 can be any appropriate machine learning algorithm, e.g., linear regression, logistic regression, Bayes classifiers, random classifiers, decision trees, and neural networks. In a particular example, the machine learning logic 254 is a boosted decision tree.

The machine learning engine 250 can be based on, e.g., a decision tree model, such as a trained boosted decision tree model. The model can be used to determine a degree (e.g., a prediction score) of a medical condition based at least in part on the brain data by, e.g., assessing whether a specific set of activations found in the connectivity matrix (e.g., the connectivity matrix determined based on the image of the brain obtained by the medical imaging system 220) is correlated with the medical condition.

Generally, boosted decision tree ensembles include a sequence of consecutive trees, at each level. The trees are trained in a consecutive way. Each individual model learns from mistakes made by the previous model. When an input is misclassified by a hypothesis, one or more weights of the trees in the ensemble are altered such that the next hypothesis is more likely to classify it correctly. Combining the whole set at the end converts weak learners into a better performing model. Any appropriate method of boosting may be used, for example gradient boosting, XGboost, ADAboost, and random forest. In an example, when dealing with small samples, Synthetic Minority Over-sampling Technique (SMOTE) can be used to artificially balance the sample and create minority class (low respondent) observations. The boosted decision tree can be used to generate outcome data 256 that characterizes a likelihood that the patient has a particular condition, e.g., medical condition, symptom, behavior, or trait.

The outcome data 256 may be associated with the training data 258 in order to train the machine learning logic 254 to identify functional data, e.g., connectivity data, associated with a particular medical condition or symptom.

In some implementations, the end user of the client device 202 can store the received Medical Image Analysis data 218 in the client device 202's memory 214 (along with other user files 216 that may already be stored in the memory 214). Memory 214 included in the end-user client device 202 and memory 244, may each include any memory or database module and may take the form of volatile or non-volatile memory including, without limitation, magnetic media, optical media, random access memory (RAM), read-only memory (ROM), removable media, or any other suitable local or remote memory component.

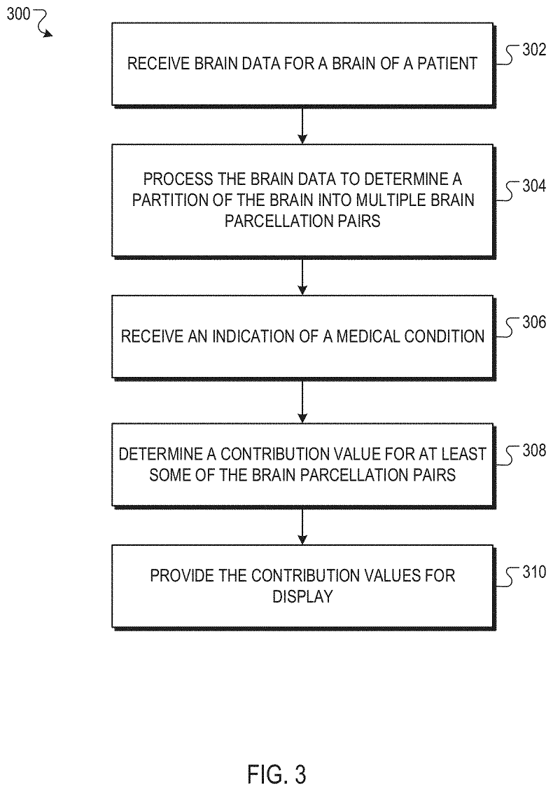

The system can further include a SHAP calculation engine 260 described in more detail below with reference to FIG. 3.

FIG. 3 is a flow diagram of an example process 300 for determining contributions of brain regions to a medical condition. The process can be implemented by a combination of hardware, software, and firmware. For example, the systems described above with reference to FIGS. 1A, 1B, and 2, can be used to perform the process 300.

The system receives brain data for the brain of a patient (302). As described above with reference to FIG. 2, a medical imaging system can obtain one or more images of the brain of the patient, and the one or more images can be processed by a machine learning model to determine processed brain data, e.g., one or more connectivity matrices that characterize functional/structural connectivity in the brain of the patient.

The system processes the brain data to partition the data into multiple brain parcellation pairs (304). For example, in a connectivity matrix, each element of the matrix can characterize the degree of connection (e.g., the degree of correlation of activity between a pair of parcellations where the column of the matrix element is assigned to a first parcellation and the row of the matrix element is assigned to a second parcellation).

The system receives an indication of a condition (306). For example, as described above with reference to FIG. 2, a user can use a graphical user interface of a computing device to provide an input that can indicate, e.g., a particular medical condition, symptom, behavior, or trait. The indication can also be received in any other appropriate manner and without specific user input. In some implementations, a machine learning model (e.g., the boosted tree model described above with reference to FIG. 2, or any other appropriate machine learning model) can process the brain data and generate a prediction (e.g., a prediction score) that characterizes a likelihood that the patient has the medical condition.

The system determines a contribution value for brain parcellation pairs to the condition, symptom or trait in question, where the contribution value for each characterized pair characterizes a contribution of the brain parcellation pair (308) to the condition. In some implementations, the contribution value is a SHAP (SHapley Additive exPlanations) value, and determining the contribution value for brain parcellation pairs includes determining the SHAP value for each brain parcellation pair characterized using the SHAP methodology. Generally, the SHAP value for a feature represents an importance of the feature for a particular prediction. In this implementation, the feature is the brain parcellation pair and the prediction is the prediction score that characterizes the likelihood that the patient has the condition (e.g., as determined by the machine learning model). A positive SHAP value can represent a positive contribution to the medical condition, and a negative SHAP value can represent a negative contribution to the medical condition (e.g., can represent a protective characteristic). The SHAP methodology can generate the SHAP values for each brain parcellation pair based on the model that determines the prediction score and based on the data that defines the brain parcellation pairs (e.g., the connectivity matrices). The inputs to a SHAP calculation engine 260 (shown in FIG. 2) include:1) the patient's brain data and 2) data identifying the machine learning model used to determine the likelihood that the patient has the condition, e.g., the medical condition, based on the patient's brain data. The output of the SHAP calculation engine is a set of SHAP values characterizing the contribution of specified parcellation pairs to the model's prediction.

Example SHAP methodology is described with reference to: Lundberg, Scott M., and Su-In Lee. “A unified approach to interpreting model predictions.” In Proceedings of the 31st international conference on neural information processing systems, pp. 4768-4777. 2017. However, the SHAP methodology is provided for illustrative purposes only, and the contribution of each brain parcellation pair to the medical condition can be determined in any other appropriate manner, e.g., by using any technique that can determine feature importance at an individual level. In one example, the contribution values can be determined by using the permutation feature importance technique. Example permutation feature importance technique is described in more detail with reference to: Breiman, Leo, “Random forests,” Machine learning 45.1, 2001. In another example, the contribution values can be determined by using the LIME (Local Interpretable Model-agnostic Explanations) technique. Example LIME technique is described in more detail with reference to: MT Ribeiro, et al., “Why should I trust you? Explaining the predictions of any classifier,” Proceedings of the 22nd ACM SIGKDD international conference on knowledge discovery and data mining, 2016.

The system provides the contribution values for display on a user computing device (310). In some implementations, the system provides a visualization that compares the respective contributions of multiple brain parcellation pairs to the medical condition based on the respective contribution values. In one example, as will be described in more detail below with reference to FIG. 4, the system can generate, e.g., a waterfall diagram based on the contribution values, and provide it for display on the user device. In particular, the waterfall diagram can show, e.g., the contribution values of parcellation pairs in the order of their absolute magnitudes.

In some implementations, the combination of the respective contribution values of all brain parcellation pairs represents a probability score that characterizes an overall likelihood of the patient having the medical condition (e.g., where the probability score is the prediction generated by the machine learning model based on the brain data). In other words, the contribution values can provide an insight into the most dominant, or most highly contributing, brain parcellation pairs to the medical condition, and explain the basis on which the machine learning model generated the prediction score in regard to the medical condition.

In some implementations, the system can provide contribution values to a user computer for display according to a criteria. For example, the criteria can specify a number of top-contributing brain parcellation pairs based on the absolute magnitudes of the contribution values, e.g., top 2, 5, 10, 50, 100, etc., brain parcellation pairs. For example, if the criteria specifies the number 10, then the system can provide 10 top-contributing brain parcellation pairs for display, based on absolute magnitudes of their contribution values. In some implementations, the criteria can instead specify the number of lowest-contributing brain parcellation pairs based on absolute magnitudes of the contribution values.

Further, in some implementations, the criteria can specify a threshold absolute magnitude contribution value, and the system can provide for display all brain parcellation pairs with contribution values having absolute magnitudes that are above (or below) the threshold. For example, if the threshold specifies a contribution value of 0.2, the system can provide for display all brain parcellation pairs that have the contribution value with absolute magnitude above 0.2 (e.g., a pair having a contribution value of 0.3, and a pair having a contribution value of −0.3). However, the above criteria are specified for illustrative purposes only, and the criteria can include any other appropriate aspect and can be specified by a user, or determined automatically by the system.

In some implementations, the system can process the brain data to determine a connectivity value for each of the brain parcellation pairs of the patient, where the connectivity value for the brain parcellation pair can characterize blood flow or blood oxygen level over time in the regions of the brain represented by the brain parcellation pair. The system can determine the connectivity value in any variety of ways. In one example, the machine learning model described above with reference to FIG. 2 can be used to determine the connectivity values based on the images of the brain of the patient.

Further, the system can determine, for each brain parcellation pair of the patient, a position of the connectivity value of the brain parcellation pair of the patient within either a first distribution of connectivity values or a second distribution of connectivity values. In particular, the first distribution of the connectivity values can be specified for a particular brain parcellation pair across a population that does have the medical condition, and the second distribution of connectivity values can be specified for the brain parcellation pair across a population that does not have the medical condition. Data defining the first and the second distributions can be predetermined and can be obtained in any variety of ways. For example, the data can be obtained from, e.g., a clinical research database that includes brain data tagged with a quantified set of one or more medical conditions, behaviors, symptoms, and/or traits, across a population.

As described in more detail below with reference to FIG. 4, by determining the connectivity value for a particular brain parcellation pair of the patient, the system can identify a position of the patient either within the first distribution or within the second distribution of the connectivity values across the population. For example, if the prediction of the machine learning model determines that the patient displays the symptom of anxiety, the top-contributing brain parcellation pair of the patient (e.g., the brain parcellation pair with the largest positive contribution value, when compared to the contribution values of the other brain parcellation pairs of the patient) for anxiety can be expected to have the connectivity value that is positioned within the distribution of connectivity values of the population that does have anxiety.

Accordingly, the system can position the connectivity value of the brain parcellation pair of the patient on a plot that represents the distributions of connectivity values of the same brain parcellation pair across the population and thereby determine where the patient stands in the context of the population and also in the context of either having the medical condition or not having the medical condition.

FIG. 4 illustrates an example graphical user interface 402 displaying a visualization of contributions of different brain regions to a condition, e.g., a medical condition, based on data provided by the medical image analysis server 240 (shown in FIG. 2). The graphical user interface 402 can be, e.g., the interface 212 described above with reference to FIG. 2 and can form a part of the system 200. A user (e.g., a clinician and/or a patient) can view the interface 402 in an end user client (e.g., the end user client 202 in FIG. 2) and interact with the client by, e.g., providing an input to the client.

In one example, the input can be brain data that includes one or more connectivity matrices characterizing a structural/functional connectivity in the brain of the patient. As described above, the brain data can be obtained through, e.g., medical imaging, or in any other appropriate manner. In particular, the brain data can include multiple brain parcellation pairs representing different regions of the brain of the patient. The user can upload brain data by interacting with the connectome upload element 404 in the interface 402.

After uploading the brain data, the user can interact with the symptom selection element 406 in the interface 402 to select a medical condition, a symptom, a behavior, or a trait, from a drop-down list. As illustrated in FIG. 4, these can include, e.g., active social avoidance, anxiety, conceptual disorganization, depression, difficulty in abstract thinking, or any other appropriate medical condition.

After receiving the brain data through the element 404 and the indication of the symptom through the element 406, the system can obtain a model from a set of models for processing brain data to predict whether the patient has the medical condition. As described above with reference to FIG. 2, a machine learning model can process the brain data and determine a prediction score that characterizes a likelihood that the patient has the medical condition. For example, as illustrated in FIG. 4, after the symptom of anxiety has been selected, the machine learning model processes the uploaded brain data of the patient and determines that the likelihood of the symptom of anxiety in this patient is 18.34% (this prediction score also corresponds to f(x)=1.21, as shown in the plot 410).

Further, the system determines contribution values for each of the brain parcellation pairs included in the uploaded brain data based at least in part on the brain data and the model (e.g., the machine learning model that generated the prediction score). The contribution values characterize the contribution of each brain parcellation pair to the symptom, e.g., to the overall prediction score of 18.34% (1.21) for anxiety determined by the machine learning model for the patient. In some implementations, as described above, the system can use the SHAP methodology to determine a respective SHAP value for each brain parcellation pair that can be positive for a positive contribution (e.g., representing a risk factor for the symptom), and negative for a negative contribution (e.g., representing a protective characteristic against the symptom).

As shown in the plot 410, the system can provide the contribution values for display on the interface 402. The plot 410 represents a waterfall diagram with x-axis having the contribution values and y-axis having different brain parcellation pairs (e.g., R_5m . . . R_LBelt is a brain parcellation pair). The waterfall diagram includes 10 brain parcellation pairs with the SHAP values having the largest absolute magnitudes, e.g., having the largest positive/negative contributions to the symptom of anxiety. The x-axis starts at a value of 0.06 because this represents the base rate of the level of anxiety in the population, e.g., there is a slight tendency towards anxiety in the population. The waterfall diagram starts at 0.06 and progressively adds/subtracts the largest/smallest contribution values until the final prediction score of 1.21 for anxiety is reached (that corresponds to the prediction score of 18.34% determined by the machine learning model).

For example, for the first brain parcellation pair (e.g., R_5m . . . ), the system starts from the base rate 0.06 and adds the contribution value of 0.64 for the first brain parcellation pair (e.g., the contribution value with the largest absolute magnitude). Next, the system adds the contribution value of −0.47 for the second brain parcellation pair (R_WMV2 . . . ) because it is the contribution value with the second largest absolute magnitude out of all brain parcellation pairs. Next, the system adds the contribution value of −0.45 for the third brain parcellation pair (R_STSva . . . ) because it is the contribution value with the third largest absolute magnitude out of all brain parcellation pairs. The system continues this process until it reaches the final prediction score of 1.21 for the symptom of anxiety in the patient. In certain embodiments, the final prediction score is given by the combination of all contribution values of all brain parcellation pairs for which a SHAP value is determined. In certain embodiments, the visualization (e.g., the waterfall diagram) can be presented for a particular number of brain parcellation pairs (e.g., 10 brain parcellation pairs with the highest absolute magnitudes of the contribution values), as desired (and the final prediction score can be shown for all parcellation pairs or just the final prediction score that is produced as a result of the displayed parcellation pairs or both final scores can be displayed).

In addition to determining the contribution values of different brain parcellation pairs of the patient to a medical condition, the system can further determine a connectivity value of a particular brain parcellation pair of the patient, and the position of the connectivity value of the patient with respect to the distribution of connectivity values in a population. As described above, the system can process the brain data of the patient uploaded through the element 404 and determine the connectivity values for each brain parcellation pair by using a machine learning model. A user can interact with the element 408 and select a particular brain parcellation pair from the drop-down list 408, e.g., the brain parcellation pair that includes brain parcels R_5m and R_LBelt. As shown in the element 406, this brain parcellation pair has the parcel to parcel connectivity value of 0.14.

In response to the input from the user, the system can generate the plot 420 that shows a first distribution of connectivity values across a population that does not have the symptom (e.g., anxiety in this example) shown in grey color, and a second distribution of connectivity values across a population that does have the symptom (e.g., anxiety in this case) shown in white color. Further, the system can plot the connectivity value of 0.14 of the selected brain parcellation pair R_5m R_LBelt on the plot 420 and thereby position the patient either in the distribution across the population that does have the symptom, or the distribution across the population that does not have the symptom. In this example, the connectivity value of 0.14 is positioned in the distribution of the population that does have the symptom. This can be compared to the contribution value of 0.64 of the parcellation pair R_5m R_LBelt for the symptom of anxiety, e.g., this brain parcellation pair has a large positive contribution to the symptom of anxiety.

This information can be useful to guide the treatment to be an activation or deactivation stimulation burst.

To produce plot 420, a reference sample is separated in two classes: those for which the SHAP values for a specific parcel show that this parcel leads to the symptom and those for which the SHAP values for a specific parcel show that this parcel does not lead to the symptom. Based on this class-separated reference sample, the activation curve can be measured based on the actual correlation values in the connectivity matrix for each class/group. The patient is then plotted on that chart. The grey boxes are a histogram representing the distribution of the activation value of the two groups created by the shap values. In some embodiments, the range of values for the activation value can be between −1 and +1. Assuming pre-processing of the brain image data, the visualizations 410 and/or 420 can be generated in less than 1 hour, less than 30 minutes, less than 10 minutes, less than 5 minutes, less than 1 minute, less than 30 seconds, less than 10 seconds or less than 5 seconds in part a function of processor speed and size of the data set.

Accordingly, the plots 410 and 420 showing the contribution scores of different brain parcellation pairs of the patient, and the position of the connectivity value of a particular brain parcellation pair of the patient within a population, respectively, can allow clinicians to break-down and interpret the overall prediction score (e.g., generated by the machine learning model) for a particular symptom. The plots 410 and 420 can allow clinicians to visualize the drivers of a symptom on the level of individual brain regions of the patient. The machine learning models that generate the prediction scores can often provide complex outputs, and visualizing this information in an easy-to-digest way is critical in order to inform clinicians and facilitate effective decision making regarding the treatment of the patient.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone, running a messaging application, and receiving responsive messages from the user in return.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method comprising:
receiving brain data for a brain of a patient;
processing the brain data to determine a partition of the data into a plurality of brain parcellation pairs;
receiving an indication of a medical condition;
determining a contribution value for at least some of the plurality of brain parcellation pairs, wherein the contribution value characterizes a contribution of the brain parcellation pair to the medical condition; and
providing the contribution values for display on a user computing device.

2. The method of claim 1, wherein the contribution value is a SHAP value, and wherein determining the contribution value for each brain parcellation pair comprises determining the SHAP value for each brain parcellation pair using SHAP methodology.

3. The method of claim 1, further comprising providing, on a user computing device, a visualization that compares the respective contributions of the plurality of brain parcellation pairs to the medical condition based on the respective contribution values.

4. The method of claim 1, further comprising:
processing the brain data of the brain of the patient to determine a connectivity value for each of the brain parcellation pairs of the patient, wherein the connectivity value for the brain parcellation pair characterizes blood flow or blood oxygen level over time in regions of the brain represented by the brain parcellation pair; and
determining, for each brain parcellation pair of the patient, a position of the connectivity value of the brain parcellation pair of the patient within either a first distribution of connectivity values or a second distribution of connectivity values.

5. The method of claim 4, wherein the first distribution of connectivity values is specified for the brain parcellation pair across a population having the medical condition, and the second distribution of connectivity values is specified for the brain parcellation pair across a population not having the medical condition.

6. The method of claim 4, further comprising providing, on a user computing device, a visualization that indicates the position of the patient within the first distribution or the second distribution.

7. The method of claim 1, wherein processing the brain data of the brain to determine the partition of the brain data into the plurality of brain parcellation pairs comprises generating a connectivity matrix that characterizes a connectivity in the brain of the patient.

8. The method of claim 1, wherein the combination of the respective contribution values of all brain parcellation pairs represents a probability score that characterizes an overall likelihood of the patient having the medical condition.

9. The method of claim 8, wherein the probability score is determined by using a trained machine learning model that is configured to process an input derived from the brain data of the brain of the patient and generate the probability score.

10. A system comprising:
one or more computers; and
one or more storage devices communicatively coupled to the one or more computers, wherein the one or more storage devices store instructions that, when executed by the one or more computers, cause the one or more computers to perform operations comprising:
receiving brain data for a brain of a patient;
processing the brain data to determine a partition of the data into a plurality of brain parcellation pairs;
receiving an indication of a medical condition;
determining a contribution value for each brain parcellation pair, wherein the contribution value characterizes a contribution of the brain parcellation pair to the medical condition; and
providing the contribution values for display on a user computing device.

11. The system of claim 10, wherein the contribution value is a SHAP value, and wherein determining the contribution value for each brain parcellation pair comprises determining the SHAP value for each brain parcellation pair using SHAP methodology.

12. The system of claim 10, further comprising providing, on a user computing device, a visualization that compares the respective contributions of the plurality of brain parcellation pairs to the medical condition based on the respective contribution values.

13. The system of claim 10, further comprising:
processing the brain data of the brain of the patient to determine a connectivity value for each of the brain parcellation pairs of the patient, wherein the connectivity value for the brain parcellation pair characterizes blood flow or blood oxygen level over time in regions of the brain represented by the brain parcellation pair; and
determining, for each brain parcellation pair of the patient, a position of the connectivity value of the brain parcellation pair of the patient within either a first distribution of connectivity values or a second distribution of connectivity values.

14. One or more non-transitory computer storage media storing instructions that when executed by one or more computers cause the one or more computers to perform operations comprising:
receiving brain data for a brain of a patient;
processing the brain data to determine a partition of the data into a plurality of brain parcellation pairs;
receiving an indication of a medical condition;
determining a contribution value for each brain parcellation pair, wherein the contribution value characterizes a contribution of the brain parcellation pair to the medical condition; and
providing the contribution values for display on a user computing device.

15. A method comprising:
receiving brain data for a brain of a patient, the brain data comprising a plurality of brain parcellation pairs;
receiving an indication of a symptom;
obtaining a model for processing the brain data to predict whether the patient has the symptom;
determining a contribution value for each brain parcellation pair in the plurality of brain parcellation pairs, wherein the contribution value characterizes a contribution of the brain parcellation pair to the symptom, the contribution value based at least in part on the brain data and the model; and
providing contribution values that meet a criteria to a user computer for display.

16. The method of claim 15, wherein the contribution value is a SHAP value, and wherein determining the contribution value for each brain parcellation pair comprises determining the SHAP value for each brain parcellation pair using SHAP methodology.

17. The method of claim 15, wherein the criteria specifies a number of top-contributing brain parcellation pairs based on magnitudes of the contribution values, and wherein the method further comprises providing the number of brain parcellation pairs and the respective contribution values to the user computer for display.

18. The method of claim 15, wherein the criteria specifies a threshold magnitude contribution value, and wherein the method further comprises providing the contribution values above the threshold, and the respective brain parcellation pairs, to the user computer for display.

19. A system comprising:
one or more computers; and
one or more storage devices communicatively coupled to the one or more computers, wherein the one or more storage devices store instructions that, when executed by the one or more computers, cause the one or more computers to perform operations comprising:
receiving brain data for a brain of a patient, the brain data comprising a plurality of brain parcellation pairs;
receiving an indication of a symptom;
obtaining a model for processing the brain data to predict whether the patient has the symptom;
determining a contribution value for each brain parcellation pair in the plurality of brain parcellation pairs, wherein the contribution value characterizes a contribution of the brain parcellation pair to the symptom, the contribution value based at least in part on the brain data and the model; and
providing contribution values that meet a criteria to a user computer for display.

20. One or more non-transitory computer storage media storing instructions that when executed by one or more computers cause the one or more computers to perform operations comprising:
receiving brain data for a brain of a patient, the brain data comprising a plurality of brain parcellation pairs;
receiving an indication of a symptom;
obtaining a model for processing the brain data to predict whether the patient has the symptom;
determining a contribution value for each brain parcellation pair in the plurality of brain parcellation pairs, wherein the contribution value characterizes a contribution of the brain parcellation pair to the symptom, the contribution value based at least in part on the brain data and the model; and
providing contribution values that meet a criteria to a user computer for display.
